(19) United States
(12) Patent Application Publication
(10) Pub. No.: US 2010/0106022 A1
(43) Pub. Date: Apr. 29, 2010

(54) CAROTID PLAQUE IDENTIFICATION METHOD

(76) Inventors: Andrew Nicolaides, Nicosia (CY); Efthyvoulos Kyriacou, Paphos (CY)

Correspondence Address: MAYER BROWN LLP, P.O. BOX 2828, CHICAGO, IL, 60690, US

(21) Appl. No.: 12/477,096

(22) Filed: Jun. 2, 2009

Related U.S. Application Data
(60) Provisional application No. 61/130,870, filed on Jun. 3, 2008.

(51) Int. Cl. A61B 8/12

(52) U.S. Cl. 600/443

(57) ABSTRACT

A method of classification of plaques using the gray level distribution of pixels in ultrasonic images of carotid plaques. The method and algorithm classifies plaques into 12 classes. Each class is associated with a different level of risk of developing symptoms.

This application claims priority to U.S. Provisional Patent Application Ser. No. 61/130,870, filed Jun. 3, 2008, which is incorporated herein by reference to the extent permitted by law.

### FIELD

Described herein are methods for use in the field of medical diagnostics. In one embodiment, a method for using ultrasonic images to identify carotid plaques associated with the development of stroke is described.

### BACKGROUND

Diseases of the cardiovascular system are an ongoing concern in many countries, including the United States as well as most countries in Europe. Such diseases are at present the leading cause of death in, for example, the United States. Among the most common of such diseases is atherosclerosis, which in part is caused by the deposits of lipoproteins resulting in arterial plaque buildup.

Atherosclerosis produces deposits of cholesterol in the arterial wall creating an abnormal narrowing of the artery called stenosis. While stenosis of the artery can be problematic in a number of areas in the body, carotid artery stenosis is one of the more potentially dangerous locations because the carotid artery is the source of oxygenated blood to the brain. Some plaques which are unstable may ulcerate or rupture discharging debris into the lumen of the carotid artery. A thrombus (clot) may subsequently form on the ulcerated surface. Debris or thrombus may be carried to and obstruct the flow of blood in arteries of the brain producing neurological symptoms such as transient monocular blindness, transient weakness of one side of the body (mini stroke) or permanent stroke when part of the brain dies.

Individuals with carotid stenosis may exhibit symptoms, or may be asymptomatic. The risk of stroke in asymptomatic patients is approximately 2% per year when the internal carotid artery stenosis is greater than 70%. Following intervention (operation or stenting) this risk is reduced to 1% per year. However, perioperative stroke and death is 2 to 3% for surgery and 3-5% for stenting.

Ultrasound is a preferred method of imaging for detection of carotid stenosis, as it is non-invasive and does not require injection of dyes or other contrast agents. However, current ultrasonic imaging, which grades the severity of stenosis, places too many patients in the high risk group of more than 2% annual stroke rate and results in many unnecessary operations. Approximately 90 operations are required to prevent one stroke in the following year. It has been argued that better imaging techniques that can identify which plaques are associated with high risk (say greater than 5%) or low risk (say less than 1%) for stroke will help a practitioner make better clinical decisions on intervention. Thus, only patients at increased risk will be subjected to intervention (operation or stenting) and many will be spared from an unnecessary procedure that is expensive and carries a significant perioperative complication rate. Under such circumstances only 25 operations will be needed to prevent one stroke in the following year.

High-resolution ultrasound provides information not only on the degree of carotid artery stenosis but also on the characteristics of the arterial wall including the size and consistency of atherosclerotic plaques. Several studies have indicated that “complicated” carotid plaques are often associated with ipsilateral neurological symptoms and share common ultrasonic characteristics, being more echolucent (weak reflection of ultrasound and therefore containing echo-poor structures) and heterogeneous (having both echolucent and echogenic areas). In contrast, “uncomplicated” plaques, which are often asymptomatic, tend to be of uniform echogenic consistency (uniformly hyperechoic) without evidence of ulceration.

In an effort to improve the reproducibility of visual (subjective) classification, a consensus conference has suggested that echodensity should reflect the overall brightness of the plaque with the term hyperechoic referring to echogenic (white) and the term hypoechoic referring to echolucent (black) plaques. The reference structure, to which plaque echodensity should be compared with, should be blood for hypoechoic, the sternomastoid muscle and for isoechoic and bone for hyperechoic plaques. More recently, a similar method has been used by Polak.

In the past a number of workers had confused echogenicity with homogeneity. It is now realized that measurements of texture are different from measurements of echogenicity. The observation that two different atherosclerotic plaques may have the same overall echogenicity but frequently have variations of texture within different regions of the plaque has been made as early as 1983. The term homogenous should therefore refer to plaques of uniform consistency irrespective of whether they are predominantly hypoechoic or hyperechoic. The term heterogenous should be used for plaques of non-uniform consistency, i.e. having both hypoechoic and hyperechoic components (Gray-Weale types 2 and 3). Although O'Donnnell had proposed this otherwise simple classification in 1985 and Aldoori in 1987, there has been considerable diversity in terminology used by others. Because of this confusion, frequently plaques having intermediate echogenicity or being complex are inadequately described. For example, echolucent plaques have been considered as heterogeneous. A reflection of this confusion is a report from the committee on standards for non-invasive vascular testing of the Joint Council of the Society for Vascular Surgery and the North American Chapter of the International Society for Cardiovascular Surgery proposing that carotid plaques should be classified as homogeneous or heterogeneous.

Regarding the clinical significance of carotid plaque heterogeneity, it seems that the heterogeneous plaques described in the three studies published in the 1980's, include hypoechoic plaques and contain hypoechoic areas (large or small) and appear to be the plaques which are associated with symptoms or if found in asymptomatic individuals they are the plaques that subsequently tend to become symptomatic.

Attempts by others using visual classification of plaques have produced variable results; also, the use of the gray scale median (GSM) after image normalization has been cumbersome. None of these methods have identified groups at high enough risk and thus have not been accepted into routine clinical practice. It would be beneficial if an automated or computerized method of identifying high risk plaques could be developed to make such identification quick, simple and accurate for the medical profession.

### SUMMARY

One embodiment described herein comprises a method of classification of plaques using the gray level distribution of pixels in ultrasonic images. In one embodiment, the method and algorithm used allows for the semi-automatic classification using a computer. Plaques can be classified into 12 classes (A to L) of which 4 are associated with high risk and the rest with low risk for stroke.

Other embodiments, objects, features and advantages will be set forth in the detailed description of the embodiments that follows, and in part will be apparent from the description, or may be learned by practice, of the claimed invention. These objects and advantages will be realized and attained by the processes and compositions particularly pointed out in the written description and claims hereof. The foregoing Summary has been made with the understanding that it is to be considered as a brief and general synopsis of some of the embodiments disclosed herein, is provided solely for the benefit and convenience of the reader, and is not intended to limit in any manner the scope, or range of equivalents, to which the appended claims are lawfully entitled.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1 is a diagrammatic representation of the algorithm which is applied on the images of plaques after they have been normalized for gray scale. It shows the sequence of the steps taken and the criteria for each step in the classification of plaques according to the distribution of the different gray levels of the pixels in the image.

FIG. 2. Example of plaque A

FIG. 3. Example of plaque F

FIG. 4. Example of plaque G

FIG. 5. Example of plaque H

FIG. 6. Example of plaque I

FIG. 7. Example of plaque J

FIG. 8. Example of plaque K

FIG. 9. Example of plaque L

### DETAILED DESCRIPTION

While the present invention is capable of being embodied in various forms, the description below of several embodiments is made with the understanding that the present disclosure is to be considered as an exemplification of the claimed subject matter, and is not intended to limit the appended claims to the specific embodiments illustrated. The headings used throughout this disclosure are provided for convenience only and are not to be construed to limit the claims in any way. Embodiments illustrated under any heading may be combined with embodiments illustrated under any other heading.

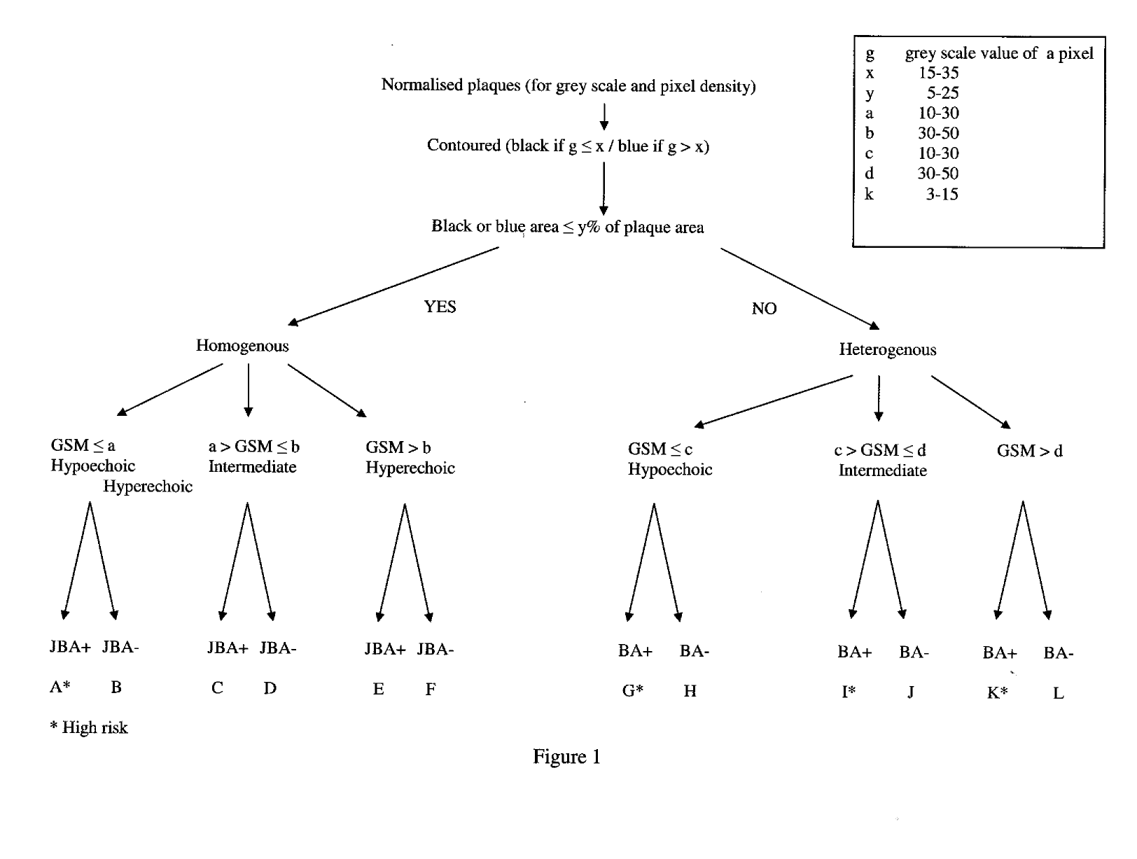

One embodiment described herein comprises an algorithm as shown in FIG. 1, which is applied on the images of plaques after said images have been normalized using blood and adventitia as reference points (blood=0 and adventitia=190). One such embodiment of the algorithm comprises some combination of the following steps:

Step 1

The image is normalized for pixel density using the bicubic method and the mm scale present in the image for distance reference. Pixel density can be in the range of about 10 to about 30 pixels per mm². However, it is possible that the density can extend beyond this range.

Step 2

The area of the plaque is outlined and saved as a separate image file.

Step 3

In one embodiment, the plaque is contoured into black and blue areas according to the gray levels of the pixels. In some embodiments, pixels with gray level equal or lower than gray value x are given the colour black and pixels with gray level greater than gray value x are given the colour blue. In various embodiments the value of x can be in the range of about 15 to about 35, although in other embodiments the value of x may be set outside that range. The choice of color is arbitrary and is purely for ease of description of the steps of the algorithm. In practice any color can be used.

Step 4

If the percentage of black or blue pixels is equal or less than y, plaques are considered to be Homogenous. If the percentage of black or blue pixels is in the range of y-(100-y), plaques are considered to be Heterogenous. In one embodiment, the value of y may fall in the range of about 5 to about 25.

Step 5

The gray scale median (GSM) of the plaque is calculated.

Step 6

If GSM is equal or less than value a the plaque is considered to be Hypoechoic. If GSM is greater than value b the plaque is considered to be Hyperechoic. If GSM is between values a and b the plaque is considered to be Intermediate. The value of a can be in the range of about 10 to about 30. The value of b can be in the range of about 30 to about 50.

Step 7

On the basis of the findings under step 6, Homogenous plaques are subclassified as Homogenous Hypoechoic, Homogenous Intermediate and Homogenous Hyperechoic.

If GSM is equal or less than value c the plaque is considered to be Hypoechoic. If GSM is greater than value d the plaque is considered to be Hyperechoic. If GSM is between values c and d the plaque is considered to be Intermediate. The value of c can be in the range of about 10 to about 30. The value of d can be in the range of about 30 to about 50. Note: the values of c and d can be different than the values of a and b.

Heterogenous plaques are similarly subclassified as Heterogenous Hypoechoic, Heterogenous Intermediate and Heterogenous Hyperechoic. In one embodiment the values of c and d used are the same as used in the homogenous classification. In another embodiment one or both values may be different than those used in the homogenous classification.

Step 8

The image is despecled. (This step may be omitted depending on the ultrasonic equipment used). Despecling is a method of image processing that removes “noise” or “smooths” the image by removing small bright or dark areas (say less than 16 pixels in size). One of skill in the art will understand that there are numerous ways of despecling that are intended to fall within the scope of this optional step of the invention.

Step 9

The size of the largest black area that is adjacent to the lumen of the artery in the absence of a visible echogenic cap (JBA) is determined. On histological examination atherosclerotic plaques have a fibrous cap consisting of collagen adjacent to the lumen (i.e. in contact with blood). The interface of blood and collagen reflects ultrasound. The fibrous cap is thick in stable plaques and thin in unstable plaques. Part of the cap may be missing in ruptured plaques and it does not exist on the surface of a thrombus attached to the surface of the plaque. On ultrasound examination a thick fibrous cap is visible as an echogenic (bright) area covering the plaque. A thin cap may not be visible on ultrasound because the energy of reflected ultrasound is weak; also it will not be seen if absent or if a thrombus which is black on ultrasound is covering the plaque. Juxtaluminal Black Area (JBA) is measured in mm² or as a percentage of the total plaque area using the pixel density from step 1. If the JBA is greater than k mm² or about m % the plaque is labeled as JBA positive (JBA+). If the JBA is equal or lower than k mm² or about m % the plaque is labeled as JBA negative (JBA−). The value of k can be in the range of about 3 to about 20 mm² and the value of m in the range of about 5% to about 30%.

Step 10

As a result of the above steps a plaques can be classified into 12 types. They are labeled A, B, C, D, E, F, G, H, I, J, K and L. (Note: The letters are arbitrary and of no significance). Plaques A, G, I and K are high risk (unstable). Plaques B, C, D, E, F, H, J and L are low risk (stable).

This algorithm may be implemented in a number of ways. For example, the algorithm may be contained within a computer program on a portable readable medium such as a disk, CD-ROM, or portable drive (such as “jump drives”, “thumb drives” or external hard drives). Such a program can then be transferred to the hard drive of a computer. In another embodiment, the algorithm is programmed into a computer or machine that is task-specific, that is, is sold as a diagnostic device with the algorithm integrated with the ultrasound device and display. Other methods of making the algorithm available for diagnostics may be used in the art and are contemplated by the present invention.

The algorithm may be supplemented by further ultrasonic, clinical or biochemical markers that may assist in more accurate determination of risk. Such markers can be those that have been shown by others to increase or decrease risk. Ultrasonic markers can be the degree of internal carotid stenosis and a number of texture features (e.g. “contrast”, “homogeneity”, presence of descrete white areas). Clinical markers can be conventional risk factors (e.g. age, gender, smoking, blood pressure, diabetes), history of contralateral (i.e. opposite side) hemispheric transient ischemic attacks or transient monocular blindness in the past or the results of other investigations such as presence of “silent” (i.e. asymptomatic) infarcts on a CT- or MRI-brain scan. Biochemical markers can be blood levels of biochemical or hematological substances (e.g. cholesterol, creatinine, hematocrit). In contrast to the level of risk determined by plaque classes A, G, I and K which is large, the effect of these biomarkers other than stenosis on risk is relatively small and for this reason they are not used in clinical decisions on intervention (operation or stenting). However, they can optionally be used to adjust (slightly increase or decrease) the risk estimated by the algorithm as a whole.

### EXAMPLES

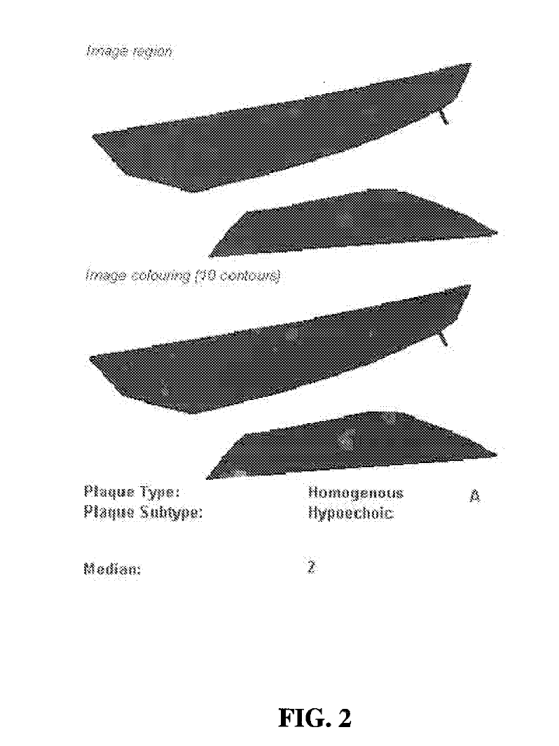

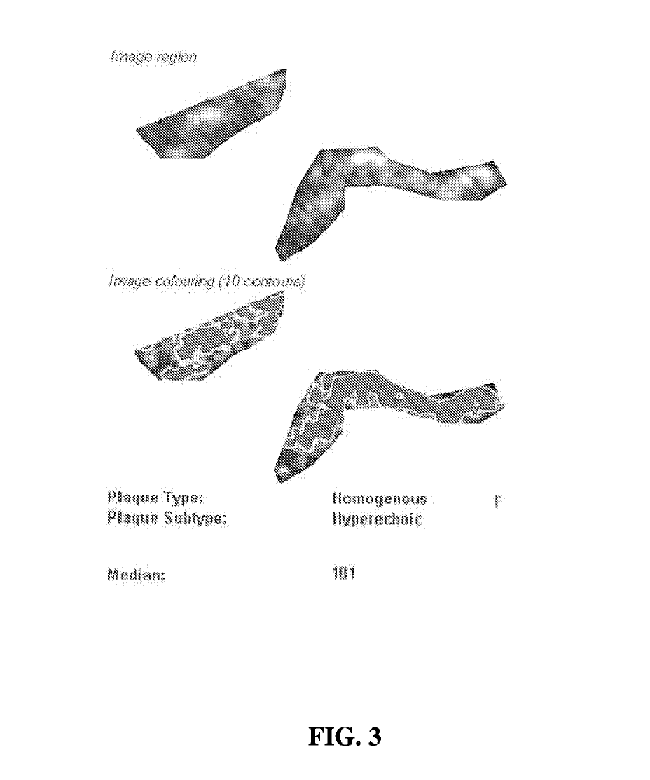

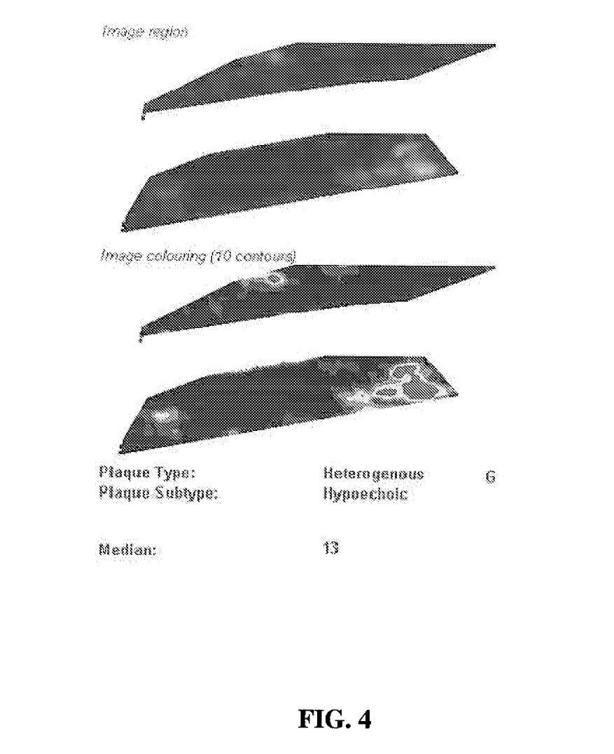

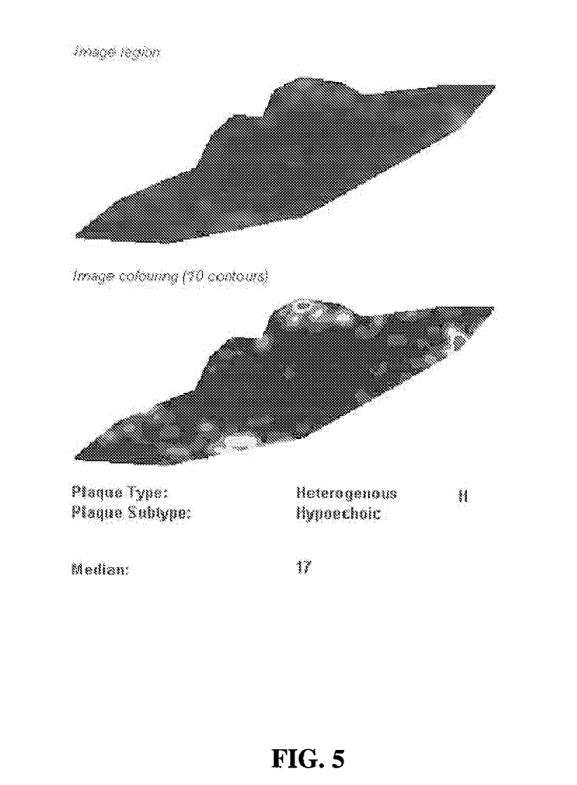

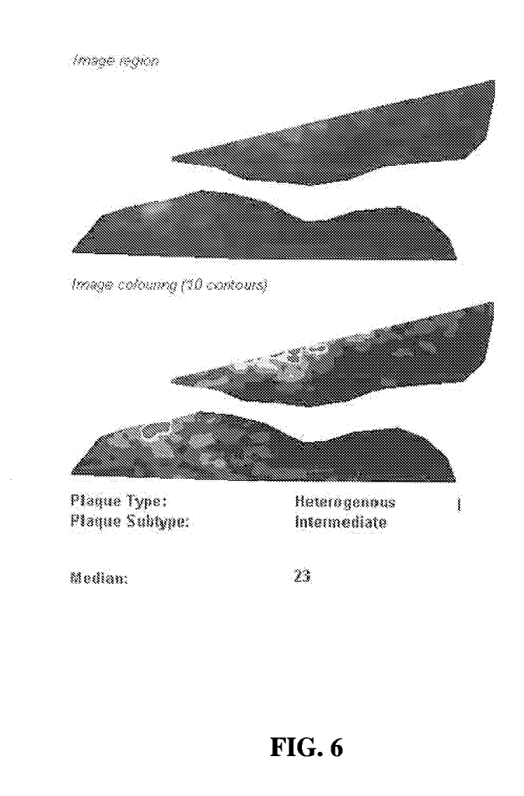

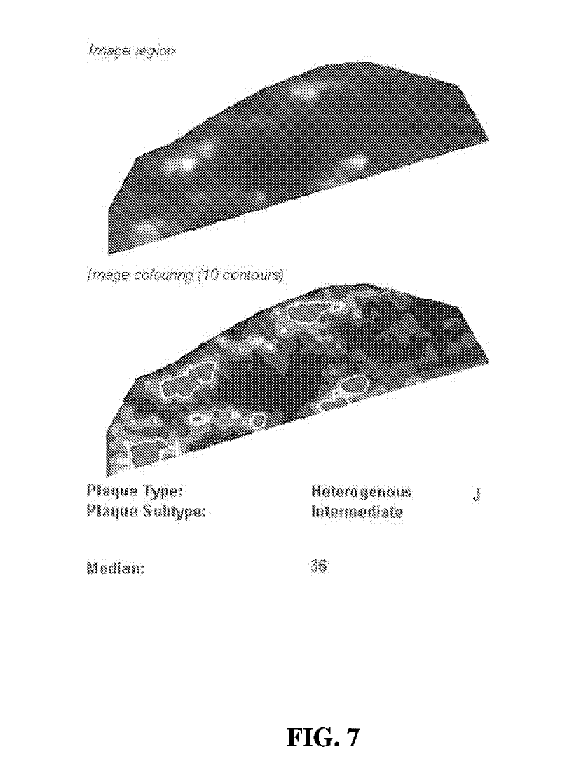

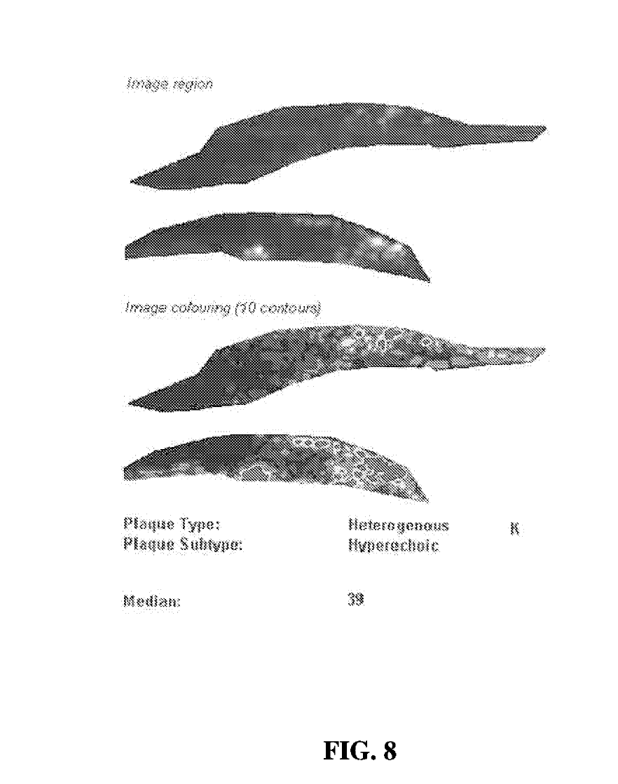

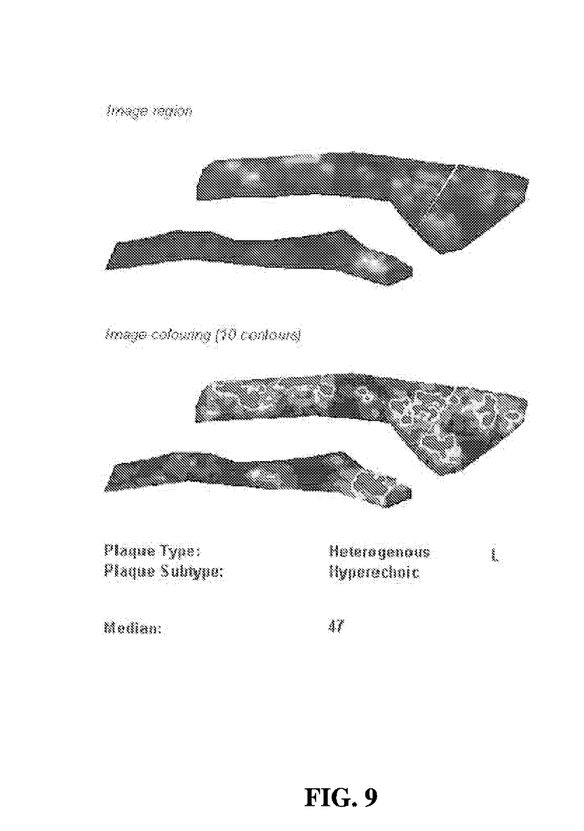

Examples of high and low risk plaques are shown in the attached FIGS. 2-9. Each plaque is shown as a black and white image and also contoured in different colors for better visual impact. In the contoured images pixels have been colored as follows:

| Gray scale | Color |
|---|---|
| 0-25 | black |
| 26-50 | blue |
| 51-75 | green |
| 76-100 | yellow |
| 100-125 | orange |
| >125 | red |

All references, including publications, patent applications, and patents, cited herein are hereby incorporated by reference to the same extent as if each reference were individually and specifically indicated to be incorporated by reference and were set forth in its entirety herein.

The use of the terms “a,” “an” and “the” and similar references in the context of this disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably) provided herein, is intended merely to further illustrate the content of the disclosure and does not pose a limitation on the scope of the claims. No language in the specification should be construed as indicating any non-claimed element as essential to the practice of the present disclosure.

Alternative embodiments of the claimed disclosure are described herein, including the best mode known to the inventors for practicing the claimed invention. Of these, variations of the disclosed embodiments will become apparent to those of ordinary skill in the art upon reading the foregoing disclosure. The inventors expect skilled artisans to employ such variations as appropriate (e.g., altering or combining features or embodiments), and the inventors intend for the invention to be practiced otherwise than as specifically described herein.

Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Thus, recitation of ranges of values herein are merely intended to serve as a shorthand method of referring individually to each separate value falling within the range, unless otherwise indicated herein, and each separate value is incorporated into the specification as if it were individually recited herein.

It is to be understood that any ranges, ratios and ranges of ratios that can be formed by, or derived from, any of the data disclosed herein represent further embodiments of the present disclosure and are included as part of the disclosure as though they were explicitly set forth. This includes ranges that can be formed that do or do not include a finite upper and/or lower boundary. Accordingly, a person of ordinary skill in the art most closely related to a particular range, ratio or range of ratios will appreciate that such values are unambiguously derivable from the data presented herein.

What is claimed is:

1. A method of identifying carotid bifurcation plaques associated with a risk level of developing symptoms, comprising the steps of:
(a). obtaining ultrasonic images of carotid plaques;
(b). normalizing the images using blood and adventitia as reference points;
(c). normalizing images to a standard pixel density; and
(d). classifying the plaques using an algorithm which uses the gray level distribution of the pixels in the ultrasonic images.

2. The method of claim 1, wherein the algorithm comprises a step of changing pixels of the ultrasonic images to one of two classes or colors based on whether their gray scale value is greater than a predetermined cut-off point.

3. The method of claim 2, wherein the predetermined cutoff point is between about 15 and about 35.

4. The method of claim 2, wherein the algorithm further comprises a step of classifying plaques according to whether the area covered by either one of the two colors is greater than a predetermined percentage of the total plaque areas.

5. The method of claim 4, wherein when the percentage is greater than the predetermined percentage the plaque is classified as heterogenous.

6. The method of claim 4, wherein when the percentage is less than or equal to the predetermined percentage the plaque is classified as homogenous.

7. The method of claim 6, wherein homogenous plaques are classified into one of three categories according to the gray scale median using two predetermined cut-off points.

8. The method of claim 5, wherein the images of the heterogenous plaques are classified into one of three categories according to the gray scale median using two predetermined cut-off points.

9. The method of claim 8, wherein the three classes are subdivided as having a juxta-luminal area of one color greater than a predetermined area in mm² without a visible echogenic cap or less than the predetermined area in mm² without a visible echogenic cap.

10. The method of claim 7, wherein the three classes are subdivided as having a juxta-luminal area of one color greater than a predetermined area in mm² without a visible echogenic cap or less than the predetermined area in mm² without a visible echogenic cap
